(19) United States
(12) Patent Application Publication
(10) Pub. No.: US 2010/0152268 A1
(43) Pub. Date: Jun. 17, 2010

(54) STABLE ATORVASTATIN FORMULATIONS

(75) Inventor: Fjalar Johannsson, Reykjavik (IS)

Correspondence Address: CANTOR COLBURN, LLP, 20 Church Street, 22nd Floor, Hartford, CT, 06103, US

(73) Assignee: ACTAVIS GROUP PTC EHF, Hafnarfjordur (IS)

(21) Appl. No.: 12/526,596

(22) PCT Filed: Feb. 11, 2008
(86) PCT No.: PCT/IS08/00005

§ 371 Date: Dec. 15, 2009

(30) Foreign Application Priority Data
Feb. 9, 2007 (IS) 8607

(51) Int. Cl. A61K 31/40; A61K 31/407; A61P 3/06

(52) U.S. Cl. 514/411; 514/423

(57) ABSTRACT

The present invention provides novel alternative pharmaceutical compositions comprising amorphous atorvastatin or a pharmaceutically acceptable salt thereof and a controlled amount of one or more additional compounds selected from the group consisting of compounds of formula I (formed by ring opening of the epoxide of Formula II to form a diketo dihydroxy derivative), II (diketo epoxide derivative), III (di-epoxide derivative formed by ring closure of the heptanoic acid chain to form a cyclohexanoate substituent), IV (2-oxo derivative with the isopropyl group rearranged to the 3-position) and V (phenantrene derivative of the 2-oxo derivative), together with one or more suitable pharmaceutical excipients, which compositions have improved stability.

### FIELD OF INVENTION

This invention relates to a pharmaceutical formulation and in particular to a stable composition for a pharmaceutical dosage form comprising atorvastatin.

### TECHNICAL BACKGROUND AND PRIOR ART

Atorvastatin ([R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid) belongs to a well known group of drugs, statins, which are useful for the treatment of hypercholesterolemia or hyperlipidemia. Statins block the hydroxyl-methylglutaryl-coenzyme A reductase (HMG-CoA), thereby specifically inhibiting cholesterol synthesis in the liver. Although cholesterol is a vital component of all cells, it also contributes to plaque formation in arteries, which plaques increase the risk for high blood pressure, heart attack and stroke. Statins stabilize the plaques, making them less prone to rupturing and subsequently forming blood cloths.

EP 247,633 discloses the synthesis of atorvastatin and its use as hypocholesterolemic agent. EP 409,281 discloses the hemi calcium salt of atorvastatin. Atorvastatin exists in multiple amorphous and crystalline forms. Originally, atorvastatin was synthesized in the amorphous form and most of the clinical pharmacology studies were conducted on such tablets. However, amorphous atorvastatin is reported to be hydroscopic and unstable when exposed to oxygen, and it has been proven difficult to develop a stable dosage form, which does not transform into different morphological forms, discolor or even degrade.

Several attempts have been made to prevent degradation of atorvastatin. For example EP 680,320 discloses pharmaceutical composition containing atorvastatin stabilized with a basic inorganic salt of magnesium, calcium or lithium.

Practical atorvastatin formulations must also be bio-available and importantly pharmaceutically active.

EP 1336405-A1 discloses formulations with amorphous atorvastatin together with inactive ingredients including lactose, microcrystalline cellulose, sodium carbonate, BHA and BHT. The application discusses the advantages of using amorphous atorvastatin with a relatively small particle size, with a d₉₀ value of less than 150 μm and a mean particle size (d₅₀) between approximately 5 and 50 μm.

WO 2006/008091 describes certain oxidative degradation products of atorvastatin calcium, indicated to be useful in order to characterize and quantify impurities and degradation in atorvastatin substance and/or pharmaceutical compositions. The goal of this disclosure is to obtain atorvastatin calcium with a high level of purity; i.e. the disclosure teaches that said compounds should preferably be non-existent in atorvastatin compositions.

As atorvastatin has proved to be an extremely useful and valuable drug, alternative formulations with increased stability and/or other pharmaceutical advantages will be highly appreciated.

### SUMMARY OF INVENTION

Accordingly, the present invention provides novel alternative pharmaceutical compositions comprising amorphous atorvastatin or a pharmaceutically acceptable salt thereof and a controlled amount of one or more additional compounds selected from the group consisting of compounds of formula I, II, III, IV and V, together with one or more suitable pharmaceutical excipients, which compositions have improved stability as shown when tested under accelerated conditions.

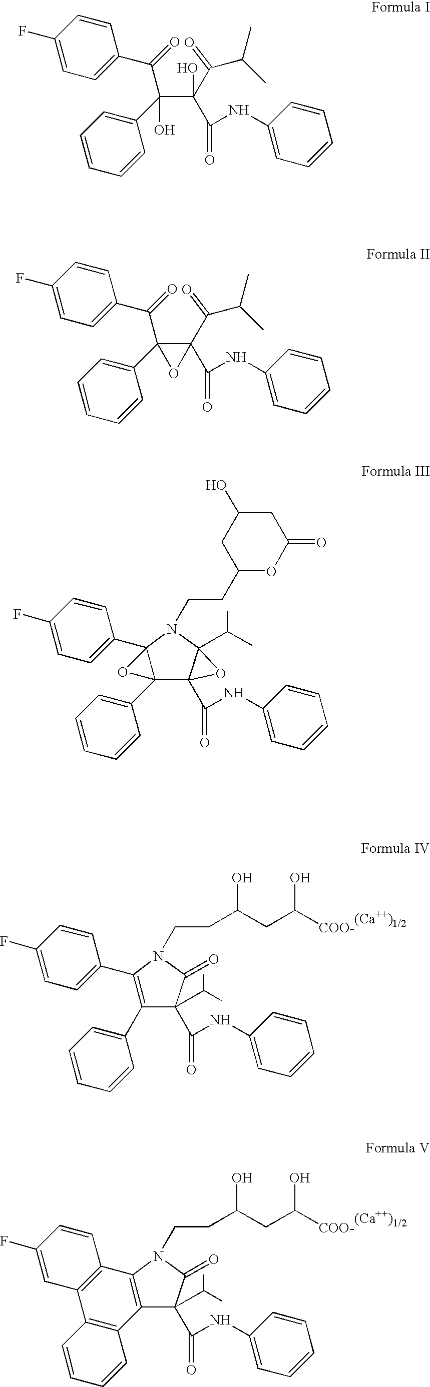

### DETAILED DESCRIPTION OF INVENTION

The chemical formula of atorvastatin hemi calcium is depicted as Formula VI below. It has been documented that the compound readily decomposes upon exposure to intense UV light (“simulated sunlight”), see Hurley et al. Tetrahedron (1984) 49, 10, pp. 1979-1984. Hurley et al. suggest that three major by-products are formed when atorvastatin dissolved in solution is exposed to UV exposure, the 2-oxo derivative with the isopropyl group rearranged to the 3-position (Formula IV; chemical name: (±)-5-(4-fluorophenyl)-2,3-dihydro-β,δ-dihydroxy-3-(1-methylethyl)-2-oxo-4-phenyl-3-[(phenylamino)carbonyl]-1H-pyrrole-1-heptanoic acid hemicalcium salt) which exists both as (−) and (+) isomer; a diketo epoxide derivative (Formula II; chemical name: 3-(4-fluoro-benzoyl)-2-isobutyryl-3-phenyl-oxirane phenyl amide) and a phenantrene derivative of the 2-oxo derivative (Formula V; chemical name: (±)-9-(fluoro-2,3-dihydro-β,δ-dihydroxy-3-(1-methylethyl)-2-oxo-3[)phenylamino)carbonyl]-1H-dibenz[e,g]indole-1-heptanoic acid hemicalcium salt) which also exists as a (−) and (+) derivative. Formulas IV and V are shown as the hemicalcium salts, but corresponding compounds may as well exist as free acids or other salts.

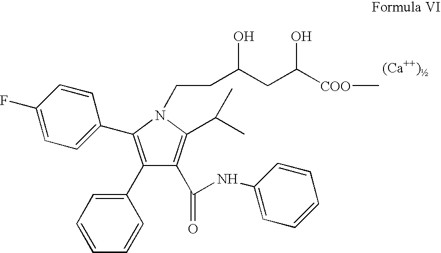

Other atorvastatin related compounds may however be formed additionally or alternatively from atorvastatin. These include a di-epoxide derivative formed by ring closure of the heptanoic acid chain to form a cyclohexanoate substituent (Formula III) (chemical name:), and a derivative closely related to the diketo epoxide derivative of Formula II (chemical name: 3-(4-fluoro-benzoyl)-2-isobutyryl-3-phenyl-oxirane-2-carboxylic acid) formed by ring opening of the epoxide to form a diketo dihydroxy derivative (Formula I).

The present inventors have surprisingly discovered that a formula of amorphous atorvastatin together with in the range of about 0, 5-5% of one or more of the above compounds I to V calculated as a weight percentage of the amount of atorvastatin and preferably with selected suitable excipients described in detail herein below, allows the production of stable tablets with excellent tablet properties and extensive shelf life. Such tablet formulations also have good bioavailability.

The mentioned compounds are suitably prepared by irradiating with a UV source a batch of atorvastatin, e.g. amorphous atorvastatin hemicalcium salt, prepared by any method well known in the art and subsequently the formed product(s) can be separated and purified e.g. by preparative HPLC.

The amorphous atorvastatin may advantageously be in the form of atorvastatin calcium, but other salts of atorvastatin can also be employed according to the present invention, such as in particular atorvastatin magnesium.

In certain embodiments the amount of said one or more compounds is in the range of about 0.5-1%. In some preferred embodiments the amount of said one or more compounds is in the range of about 1-5%, such as in the range of about 1, 2-4% or in the range of about 1, 5-4% and preferably in the range of about 1, 5-3%, calculated as described above.

It will be understood that the compositions of the invention generally comprise one or more suitable pharmaceutical excipients selected from diluents, binders, antioxidants, surfactants, disintegrants, lubricants and glidants.

In particular, it has been proven useful to include in the compositions an antioxidant, such as but not limited to butylated hydroxyanisole (BHA), butylated hydroxytoluene (BHT), ascorbic acid or a salt thereof, a sulfatide salt, citric acid, propyl gallate, alfa-tocopherol, and ascorbyl palmitate. Depending on the selected antioxidant compound, a suitable amount is e.g. in the range of about 0.1-0.5 wt %, such as in the range of 0.1-0.3 wt %.

In one embodiment the formulation comprises in the range of 30 to 85% of a filler substance, which may be one of numerous substances generally known in the art, such as e.g. a saccharide (e.g. lactose or mannitol) or microcrystalline cellulose or a mixture thereof, and may further comprise in the range of 0.5 to 5 wt % of a disintegrant such as crosslinked sodium carboxymethylcellulose (NaCMC), crosslinked polyvinylpyrrolidone or mixtures thereof. Suitable binders include povidone (2-pyrrolidinone), hydroxypropyl cellulose, pregelatinized starch, gelatin and mixtures thereof. The formulations may additionally comprise other commonly used excipients that are compatible with the active ingredient such as pigments, colorants, sweeteners, taste-masking agents and the like.

Suitable lubricants include one or more of magnesium stearate, sodium stearyl fumarate, stearic acid, colloidal anhydrous silica, synthetic aluminum silicate, magnesium oxide, calcium stearate, talc, hydrogenated castor oil, glyceryl dibehenate and mixtures thereof. Lubricant in the amounts varying from about 0.1% to about 4% by weight, preferably from about 0.5% to about 2% by weight. In preferred embodiments, magnesium stearate is selected as the most preferred lubricant.

It is also found useful to include in certain embodiments an alkali metal additive, for example. one or more of the compounds sodium carbonate, sodium bicarbonate, sodium phosphate, sodium dihydrogen phosphate, disodium hydrogen phosphate disodium hydrogen orthophosphate, sodium silicate, calcium carbonate, calcium hydroxide, magnesium carbonate, magnesium hydroxide and magnesium silicate. Sodium carbonate is particularly preferred.

### EXAMPLES

### Example 1

A number of formulations have been prepared and tablets compressed with the following composition as shown in Table 1, were all values indicate mg weight of component pre tablet (total weight of tablet 120 mg).

| Formulation: | 1 | 2 | 3 | 4 | 5 | 6 | 7 |
|---|---|---|---|---|---|---|---|
| Atorvastatin | 10.36 | 10.36 | 10.36 | 10.36 | 10.36 | 10.36 | 10.36 |
| calcium (amorphous) | | | | | | | |
| Mannitol | 68.48 | 68.12 | 68.12 | 68.12 | 68.72 | 63.32 | 57.32 |
| Sodium carbonate | 6.60 | 6.60 | 6.60 | 6.60 | 6.60 | 12.00 | 18.00 |
| (anhydrous) | | | | | | | |
| Microcrystalline | 18.00 | 18.00 | 18.00 | 18.00 | 18.00 | 18.00 | 18.00 |
| cellulose | | | | | | | |
| Crospovidone | 12.00 | 12.00 | 12.00 | 12.00 | 12.00 | 12.00 | 12.00 |
| Povidone | 2.40 | 2.40 | 2.40 | 2.40 | 2.40 | 2.40 | 2.40 |
| Magnesium stearate | 1.92 | 1.92 | 1.92 | 1.92 | 1.92 | 1.92 | 1.92 |
| BHA | 0.24 | 0.60 | | | | | |
| Methionine | | | 0.60 | | | | |
| Sodium ascorbate | | | | 0.60 | | | |

Additional formulations were prepared varying the amount of the antioxidant, having either 0.12 or 0.24 mg of antioxidant (BHA, methionine or sodium ascorbate).

Stability tests showed that the tablets have excellent stability and the amount of compounds from the group of compounds of formula I, II, III, IV and V remains stable.

### Example 2

The following compounds are admixed and tabletted with wet granulation using water as the granulation liquid:

Tablet formulation 2a

| | mg amount in |
|---|---|
| Ingredients | 10 mg tablet |
| Magnesium stearate | 1.92 |
| Povidone | 2.4 |
| Sodium carbonate anhydr. | 6.6 |
| Polyplasdone XL. | 12 |
| Cellulose microcrystalline | 18 |
| Mannitol 60, Ph Eur | 68.3 |
| Atorvastatin calcium (amorph) | 10.7 |
| Atorvastatin derivative of Formula I | 0.08 |
| TOTAL | 120 mg |

Tablet formulation 2b-e

| | mg amount in |
|---|---|
| Ingredients | 10 mg tablet |
| Magnesium stearate | 1.92 |
| Povidone | 2.4 |
| Sodium carbonate anhydr. | 6.6 |
| Polyplasdone XL. | 12 |
| Cellulose microcrystalline | 18 |
| Mannitol 60, Ph Eur | 68.3 |
| Atorvastatin calcium (amorph) | 10.7 |
| Calcium salt of derivative II, III, IV or V* | 0.08 |
| TOTAL | 120 mg |

*compounds of Formula IV or V can as well be hemi-Mg or Na.

1. A pharmaceutical composition comprising atorvastatin or a pharmaceutically acceptable salt thereof, one or more suitable pharmaceutical excipient and one or more compounds selected from the group consisting of the compounds of formula I, II, III, IV and V.

2. The pharmaceutical composition of claim 1 comprising amorphous atorvastatin or an amorphous form of a pharmaceutically acceptable salt thereof.

3. The pharmaceutical composition of claim 1 comprising amorphous atorvastatin calcium.

4. The pharmaceutical composition of claim 1 comprising amorphous atorvastatin magnesium.

5. The pharmaceutical composition of claim 1 wherein said one or more compounds is selected from the group consisting of the compound of formula I, II and III.

6. The pharmaceutical composition of claim 5 comprising the compound of Formula I.

7. The pharmaceutical composition of claim 1 wherein the amount of said one or more compounds is in the range of about 0.5 to 5 wt % of the total amount of atorvastatin.

8. The pharmaceutical composition of claim 7 wherein the amount of said one or more compounds is in the range of about 0.5 to 1 wt % of the total amount of atorvastatin.

9. The pharmaceutical composition of claim 1 wherein said one or more suitable pharmaceutical excipient are selected from the group consisting of diluents, binders, antioxidants surfactants, disintegrants, lubricants and glidants.

10. The pharmaceutical composition of claim 9 comprising an antioxidant selected from the group consisting of butylated hydroxyanisole (BHA), butylated hydroxytoluene (BHT), ascorbic acid or a salt thereof, a sulfatide salt, citric acid, propyl gallate, alfa-tocopherol and ascorbyl palmitate.

11. The pharmaceutical composition of claim 10 further comprising an alkali metal salt additive.

12. The pharmaceutical composition of claim 11 wherein said alkali metal salt additive comprises one or more of sodium carbonate, sodium bicarbonate, sodium phosphate, sodium dihydrogen phosphate, disodium hydrogen phosphate, disodium hydrogen orthophosphate, sodium silicate, calcium carbonate, calcium hydroxide, magnesium carbonate, magnesium hydroxide and magnesium silicate.

13. The pharmaceutical composition of claim 12 where said alkali metal salt additive comprises sodium carbonate.

14. The pharmaceutical composition of claim 1 formulated as a tablet.

15. The pharmaceutical composition of claim 1 formulated as a capsule.
